SEQUENCE LISTING (19 sequences)

SEQ ID NO: 1 - DNA, 20 bp, Artificial Sequence
  Synthetic sequence; 20 guanine nucleotides
gggggggggggggggggggg

SEQ ID NO: 2 - DNA, 19 bp, artificial sequence
  Synthetic sequence; 19 guanine nucleotides
ggggggggggggggggggg

SEQ ID NO: 3 - DNA, 18 bp, artificial sequence
  Synthetic sequence; 18 guanine nucleotides
gggggggggggggggggg

SEQ ID NO: 4 - DNA, 17 bp, artificial sequence
  Synthetic sequence; 17 guanine nucleotides
ggggggggggggggggg

SEQ ID NO: 5 - DNA, 16 bp, artificial sequence
  Synthetic sequence; 16 guanine nucleotides
gggggggggggggggg

SEQ ID NO: 6 - DNA, 15 bp, artificial sequence
  Synthetic sequence; 15 guanine nucleotides
ggggggggggggggg

SEQ ID NO: 7 - DNA, 14 bp, artificial sequence
  Synthetic sequence; 14 guanine nucleotides
gggggggggggggg

SEQ ID NO: 8 - DNA, 13 bp, artificial sequence
  Synthetic sequence; 13 guanine nucleotides
ggggggggggggg

SEQ ID NO: 9 - DNA, 12 bp, artificial sequence
  Synthetic sequence; 12 guanine nucleotides
gggggggggggg

SEQ ID NO: 10 - DNA, 11 bp, artificial sequence
  Synthetic sequence; 11 guanine nucleotides
ggggggggggg

SEQ ID NO: 11 - DNA, 10 bp, artificial sequence
  Synthetic sequence; 10 guanine nucleotides
gggggggggg

SEQ ID NO: 12 - DNA, 27 bp, artificial sequence
  Synthetic sequence; one example of the inverted repeat
agaccggggacttatcatccaacctgt

SEQ ID NO: 13 - DNA, 20 bp, Tetrahymena
gggttgggttgggttgggtt

SEQ ID NO: 14 - DNA, 29 bp, artificial sequence
  Synthetic sequence; primer for G quadruplex
ctgcacagaacaaaaacctgcaggaaacg

SEQ ID NO: 15 - DNA, 70 bp, artificial sequence
  Synthetic sequence; template G-quadruplex control
gctttcgacatgattgggttgggttgggttgggtttatcttcgtttcctgcaggtttttgttctgtgcag

SEQ ID NO: 16 - DNA, 61 bp, artificial sequence
  Synthetic sequence; template G11
gctttcgacatgattgggggggggggtatcttcgtttcctgcaggtttttgttctgtgcag

SEQ ID NO: 17 - DNA, 62 bp, artificial sequence
  Synthetic sequence; template G12
gctttcgacatgattggggggggggggtatcttcgtttcctgcaggtttttgttctgtgcag

SEQ ID NO: 18 - DNA, 63 bp, artificial sequence
  Synthetic sequence; template G13
gctttcgacatgattgggggggggggggtatcttcgtttcctgcaggtttttgttctgtgcag

SEQ ID NO: 19 - DNA, 64 bp, artificial sequence
  Synthetic sequence; template G14-412
gctttcgacatgattggggggggggggggtatcttcgtttcctgcaggtttttgttctgtgcag

(19) United States
(12) Patent Application Publication
(10) Pub. No.: US 2017/0283793 A1
(43) Pub. Date: Oct. 5, 2017

(54) DNA SEQUENCE AND A MUTATOR INSERTION SEQUENCE FOR INCREASING MUTATION RATE

(71) Applicant: ACADEMIA SINICA, TAIPEI (TW)

(72) Inventors: JUN-YI LEU, TAIPEI (TW); MICHAEL J. MCDONALD, CAMBRIDGE, MA (US)

(21) Appl. No.: 15/331,546

(22) Filed: Oct. 21, 2016

Related U.S. Application Data
(60) Provisional application No. 62/244,478, filed on Oct. 21, 2015.

(51) Int. Cl. C12N 15/10; C12N 15/11

(52) U.S. Cl. CPC C12N 15/1024; C12N 15/11

(57) ABSTRACT

The invention relates to a DNA sequence for increase of a mutation rate over a specific region of DNA. Particularly, the invention provides a unique guanine nucleotide sequence and a mutator insertion sequence incorporated with the guanine nucleotide sequence and their applications in increasing a mutation rate.

### FIELD OF THE INVENTION

The invention relates to a DNA sequence for increase of a mutation rate over a specific region of DNA. Particularly, the invention provides a unique guanine nucleotide sequence and a mutator insertion sequence incorporated with the guanine nucleotide sequence and their applications in increasing a mutation rate.

### BACKGROUND OF THE INVENTION

The extensive sequencing of cancerous cells has revealed genomes scarred by mutation. While some classes of cancer are dominated by mutation events that bear the signatures of mutagens, in others, variation manifests as unevenly distributed clusters of snps and indels which may be due to inherent, regional differences in mutation rate. Such mutation rate heterogeneity has proven to be a confounding factor in the major undertaking to distinguish cancer-causing “driver” mutations from non-causal “passenger” mutations. Such studies typically proceed under the assumption that mutations are rare and occur with equal probability at all loci in the genome. In this scenario, if the same gene is mutated across multiple cancer samples, then that gene is likely to be essential for cancer development. However, if there is variation in mutation rate across a genome, then mutations repeatedly found in a gene with a high mutation rate could be incorrectly attributed a causal role. Indeed, a recent study incorporating mutational heterogeneity into such an analysis found that most of the genes previously designated as drivers had been mistakenly assigned. While this has been especially apparent in analyses of cancer genomes, the same assumptions go into analyses of pathogenic and experimental populations. It is therefore essential that the causes of mutation rate heterogeneity be understood so that patterns of genetic variation can be correctly attributed as likely due to either selection for functional convergence or to mutation rate variation.

The factors established as having the strongest effects on genome-wide mutation rates are transcription and DNA replication timing, processes that interact intimately with DNA on a global scale. Primary DNA sequence can also influence mutation rate. It has long been appreciated that homopolymeric repeats of nucleotides are prone to increase and decrease in length at a high frequency, and this has been found to play an important role in genetic switching mechanisms, or phase variation, in pathogenic bacteria [Mirkin S M (2007) Expandable DNA repeats and human disease. Nature 447: 932-940]. A more recent discovery is that sequences that are prone to double-strand breaks [Saini N, Zhang Y, Nishida Y, Sheng Z, Choudhury S, et al. (2013) Fragile DNA motifs trigger mutagenesis at distant chromosomal loci in Saccharomyces cerevisiae. PLoS Genet 9: e1003551], can also cause mutation at a distance. For instance Tang and colleagues [Tang W, Dominska M, Gawel M, Greenwell P W, Petes T D (2013) Genomic deletions and point mutations induced in Saccharomyces cerevisiae by the trinucleotide repeats (GAA.TTC) associated with Friedreich's ataxia. DNA Repair (Amst) 12: 10-17] found that long repeats (230 triplets) but not short repeats (20 triplets) were able to induce large deletions in a reporter gene more than a kilobase downstream. Others have found that fragile 70 DNA sites, typically perfect inverted repeats of between 320 bp and 1.2 kb long, induced double strand breaks in sequences up to 8 kb away [Saini N, Zhang Y, Nishida Y, Sheng Z, Choudhury S, et al. (2013) Fragile DNA motifs trigger mutagenesis at distant chromosomal loci in Saccharomyces cerevisiae. PLoS Genet 9: e1003551.].

In previous work, it was found that short repeat sequences are positively correlated with the substitution rate in the surrounding DNA sequence [McDonald M J, Wang W C, Huang H D, Leu J Y (2011) Clusters of Nucleotide 512 Substitutions and Insertion/Deletion Mutations Are Associated with Repeat Sequences. Plos Biology 9], distinct from the well known repeat length polymorphism associated with repetitive DNA sequences, and that the experimental insertion of repeat sequences could elevate mutation rates in the downstream sequence.

Mutation results in new DNA sequences. The rate at which new mutations occur is a fundamental constraint on evolutionary processes. One of the goals of industry is to find new DNA sequences that encode proteins or organisms or value. It is often useful then to increase the rate at which new mutations occur, so that more new sequences can be produced. However, mutations occur randomly across all the genes that an organism has, not in the gene of interest, which has unpredictable and usually deleterious effects. An important goal of commercial efforts to engineer and evolve novel proteins, DNA sequences and whole organisms is to focus the increased mutation rate on a specific region of DNA. Therefore, there remains a need to develop a short repeat sequence to increase the mutation rate of a gene to engineer and evolve novel proteins.

### SUMMARY OF THE INVENTION

The invention investigates the evolutionary implications of these mutagenic DNA sequences in genomes, demonstrate which DNA replication repair pathways are necessary for mutagenesis and show these sequences interact with other known causes of mutation rate variation. The invention surprisingly found that homopolymeric runs of nucleotides base pairs of longer cause increases in the substitution rate downstream of the repeat sequence. The invention provides at least two applications. First, this invention can be used during the directed evolution of novel proteins, focusing evolutionary progress entirely on the gene or genes of interest. Secondly, the incorporation of the sequence(s) into a “mutator insertion sequence” would facilitate high throughput insertion of the sequences in genomes.

The invention provides a DNA sequence, comprising a short repeat nucleotide sequence of less than 20 guanine or adenine. In some embodiments of the invention, the short repeat nucleotide sequence has 20, 19, 18, 17, 16, 15, 14, 13, 12, 11 or 10 guanine nucleotides (respectively corresponding to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11); preferably, 11, 12, 13 or 14 guanine nucleotides (respectively corresponding to SEQ ID NO: 10, SEQ ID NO: 9, SEQ ID NO: 8 and SEQ ID NO: 7); more preferably, 14 guanine nucleotides (SEQ ID NO: 7). In a further embodiment, the DNA sequence further comprises an inverted repeat flanking one or two ends of the sequence.

The invention also provides a recombinant DNA sequence, comprising a polynucleotide sequence of interest and a DNA sequence as disclosed herein, wherein the DNA sequence is integrated into an upstream site of the polynucleotide sequence of interest.

The invention also provides a mutator insertion sequence integrated into one or more the DNA sequence as disclosed herein. In some embodiments of the invention, the mutator insertion sequence comprises a ccdB gene and a DNA sequence of as disclosed herein, wherein the DNA sequence inserts into the ccdB gene at a site 30 bp from the end of the gene. In a further embodiment, the ccdB gene is further followed by another DNA sequence as disclosed herein and another 30 bp of sequence encoding the last 10 amino acids of the ccdB protein, but using alternative codons. In another further embodiment, the mutator insertion sequence further comprises one or more repeat sequence and optional one or more restriction enzyme sites flanking one or two ends of the open reading frame of interest of the mutator insertion sequence. Preferably, the restriction enzyme site is mme1 restriction enzyme site.

The invention further provides a vector comprising the DNA sequence or a mutator insertion sequence of the invention as disclosed herein.

The invention also further provides a method for increasing a mutation rate, comprising integrating a DNA sequence, a recombinant DNA sequence, or a mutator insertion sequence of the invention as disclosed herein into a target gene of interest.

### BRIEF DESCRIPTION OF THE DRAWING

FIGS. 1 A and 1 B show experimental approach to quantifying mutagenicity of G13+ DNA sequences. FIG. 1A, Poly G sequences were engineered into position 4 base pairs upstream of the URA3 translation start site (the 5′ UTR region). The G14-URA3 construct (G14-ORF) allowed detection of loss-of-function 101 mutations in the URA3 reading frame by our mutation trap assay. The red asterisk indicates the mutation site. FIG. 1B, Using a weak URA3 allele (URA3-w), this construct (G14-repeat) facilitated the detection of the polyguanine repeat expansion mutation, as the mutation, G14 to G15 or longer, results in the 5′FoA resistant phenotype.

FIGS. 2 A to 2 D show that polyguanine sequences cause a localized, directional effect on mutation rate. FIG. 2A, Mutation rates of homopolymeric guanine repeat sequences of increasing length. The estimated phenotypic mutation rate of G0-URA3 is 5.4×10⁻⁷, G13- is 13.5×10⁻⁷, G14-URA3 is 20.3×10⁻⁷. G11 and less had no detectable increase in mutation rate (data not shown). FIG. 2B, G14 sequences do not increase global mutation rates. Mutation rate was measured for a ClonNAT resistance reporter gene (see Materials and Methods) that is not linked to the G13+ repeat sequence in the G0, G13 and G14 strains. This result supports the conclusion that G13+ sequences are associated with a local increase in mutation rate, but are not associated with a genome-wide increase in mutation rate. FIG. 2C, The G14 repeat does not cause an increase in mutation rate if engineered on the template strand (C14) or on either the coding or template strand downstream of the URA3 terminator sequence. FIG. 2D, The distribution of mutations in independent 5-FOA resistant ura3 mutants for the G0 (blue) and G14 strains (orange). The distributions are not different from each other (Mann-Whitney U, U=112, p<0.01).

FIGS. 3 A to 3 E shows that polyguanine sequences are depleted from eukaryotic genomes and associated with high levels of nucleotide substitutions. FIG. 3A, Left box, the proportion of A, T, C and G nucleotides that comprise the yeast, fly and human genomes. Right box, the proportions of A, T, C and G homopolymeric repeat sequences of length 10 bp or more. FIG. 3B, the normalized distribution of A and G repeats of length 10 nucleotides or longer in the human genome, C and T repeats not shown for clarity. FIG. 3C, The number of substitutions per DNA sequence window, with increasing distance from the A13+ (green), and G13+ (black) repeats in coding sequence in humans, and FIG. 3D, non-coding sequence in Humans. FIG. 3E, the number of indels per sequence window in the sequence surrounding G13+and A13+ repeats. The number of substitutions or indel is calculated for each sequence window (see Methods).

FIGS. 4 A and 4 B show that the effect of G14 mutagenicity is correlated with DNA replication timing. FIG. 4A, Mutation rates (open columns) of G14 sequences at four different sites on chromosome XII and FIG. 4B, chromosome XV. Turquoise circles show replication timing of each site after the release of cells into synchronized S phase, in minutes.

FIGS. 5 A to 5 C show that expansion of polyguanine repeats occurs at a much higher frequency and depends on the homologous recombination pathway. FIG. 5A, Mutation rates of G0 and G14-ORF strains compared to their respective rev1 deletion mutants. FIG. 5B, Relative mutation rates for G0, G14-repeat, and G14-ORF strains. FIG. 5C, in each box, the mutation rates of deletion mutants are shown relative to their respective mutants, either G0, G14-repeat, or G14-ORF. Significance (indicated by asterisks) is determined by non-overlapping error bars, which are 95% confidence intervals.

FIG. 6 shows model for the outcome of G13+-induced replication fork stalling. I. The replication fork stalls at G14 sequence during DNA replication. II. The replication fork detaches from template and reinitiates replication downstream leaving a patch of single stranded DNA 800-3000 bp in length. III. The DNA complementary to the single stranded gap is synthesized using either Rad52-dependent homologous recombination (detected using the G14-repeat construct) or Rev1-dependent translesion synthesis (detected using the G14 construct) to bypass the difficult-to-replicate region.

FIGS. 7 A and 7 B show that expansion of the polyguanine repeat (G14 to G15) reduces the Ura3 protein abundance but not the mRNA level. FIG. 7A, The URA3 gene was tagged with GFP in the G14-repeat and G15-repeat strains. Ura3-GFP intensity was measured using the fluorescence activated cell sorter. FIG. 7B, mRNA levels were measured using quantitative PCR.

FIGS. 8 A to 8 C show that G 11-14 sequences do not form G-quadruplex structure. FIG. 8A, In order to test for the formation of a structure that could explain the differences in mutation rate observed, we analyzed the structures formed by G11, G12, G13 and G14 oligos incubated in the presence of two ions, K+ and Na+. Incubating potential G-quadruplex quadruplex forming oligos in the presence of either Na+ or K+ ions leads to the formation of different structures which can be detected by circular dichroism. K+ 459 is the preferred ion and leads to conformationally distinct, stable structures with higher peaks. The peaks observed for the control G-quadruplex structure showed a distinct increase in stability in the presence of K+ ions compared to Na+462 , recapitulating the results of previous work using this same G463 quadruplex [Dexheimer T S, Sun D, Hurley L H (2006) Deconvoluting the structural and drug-recognition complexity of the G-quadruplex-forming region upstream of the bcl-2 P1 promoter. J Am Chem Soc 128: 5404-5415.]. However, the G11-14 sequences all showed a lesser peak than the control G-quadruplex, showing no consistent differences between different lengths of G (G11 formed just as high a peak as G14), and K+ ions did not induce a different or more stable structure compared to Na+ ions. We found these combined results strongly suggestive the G-quadruplexes are not the causative agent of G13+ induced mutagenesis. FIG. 8B, G11 -14 sequences do not stop DNA polymerase from DNA synthesis, while G-quadruplex does. DNA polymerase stop assays were performed on templates containing either a known G quadruplex forming sequence or homopolymeric G repeats of 11-14 nucleotides. The G-quadruplex forming sequence acts a positive control (lanes labeled “+”), showing that G-quadruplex formation blocks DNA synthesis in this assay. The templates containing G11-14 were synthesized across, supporting that these sequences do not form G-quadruplex structures. The assay was carried out at 37° C. and 55° C. to test for potential heat lability of structures. FIG. 8C, In yeast genomes, the sequence flanking predicted G-quadruplexes are not enriched in nucleotide diversity (n=898).

### DETAILED DESCRIPTION OF THE INVENTION

Unless specifically defined or described differently elsewhere herein, the following terms and descriptions related to the invention shall be understood as given below.

The use of terms “a” and “an” and “the” and similar referents in the context of describing the invention (especially in the context of the following claims) are to be construed to cover both the singular and the plural, unless otherwise indicated herein or clearly contradicted by context.

The term “nucleotide” refers to one monomer in a polynucleotide. A nucleotide sequence refers to the sequence of bases in a polynucleotide.

The term “polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “target site” or “target sequence” is a nucleic acid sequence that defines a portion of a nucleic acid to which a binding molecule will bind, provided sufficient conditions for binding exist.

The term “nucleic acid fragment of interest” or “polynucleotide sequence of interest” refers to any nucleic acid fragment that one wishes to insert into a genome. Examples of nucleic acid fragments of interest include any genes, such as therapeutic genes, marker genes, control regions, trait-producing fragments, and the like.

The term “coding sequence” refers to a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A transcription termination sequence may be located 3′ to the coding sequence. Other “control elements” such a regulatory sequences, e.g., promoter sequences may also be associated with a coding sequence.

The term “open reading frame” is abbreviated ORF and refers to a sequence of nucleotides in DNA that contains no termination codons and so can potentially translate as a polypeptide chain.

The term “transposase” means an enzyme that is capable of forming a functional complex with a transposon end-containing composition (e.g., transposons, transposon ends, transposon end compositions) and catalyzing insertion or transposition of the transposon end-containing composition into the double-stranded target DNA with which it is incubated in an in vitro transposition reaction.

A “DNA sequence” refers to the polymeric form of deoxyribonucleotides (adenine, guanine, thymine, or cytosine) in either single stranded form or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences.

The term “recombinant” refers to an artificial combination of two otherwise separated segments of sequence, e.g., by chemical synthesis or by the manipulation of isolated segments of nucleic acids by genetic engineering techniques.

The term “recombinant polynucleotide” is defined as a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “host cell” refers to a living cell into which a heterologous polynucleotide sequence is to be or has been introduced. The living cell includes both a cultured cell and a cell within a living organism. Means for introducing the heterologous polynucleotide sequence into the cell are well known, e.g., transfection, electroporation, calcium phosphate precipitation, microinjection, transformation, viral infection, and/or the like. Often, the heterologous polynucleotide sequence to be introduced into the cell is a replicable expression vector or cloning vector. In some embodiments, host cells can be engineered to incorporate a target gene on its chromosome or in its genome.

By “integration” it is meant that the gene of interest is stably inserted into the cellular genome, i.e., covalently linked to the nucleic acid sequence within the cell's chromosomal DNA.

The invention solves the problem known in the art that mutations occur randomly and unpredictable across all the genes by proscribing a specific and unique DNA sequence (consecutive Guanine nucleotides) that increases the mutation rate over a specific region of DNA, downstream of the repeat. The ability of this DNA sequence to cause a local increase in mutation rate distinguishes it from other methods of mutation rate manipulation that affect the whole organism.

In one aspect, the invention provides a DNA sequence, comprising a short repeat nucleotide sequence of less than 20 guanine or adenine.

In some embodiments, the DNA sequence comprises 20, 19, 18, 17, 16, 15, 14, 13, 12, 11 or 10 guanine nucleotides (respectively corresponding to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11). Preferably, the DNA sequence comprises 11 (G11), 12 (G12), 13 (G13) or 14 (G14) guanine nucleotides (respectively corresponding to SEQ ID NO: 10, SEQ ID NO: 9, SEQ ID NO: 8 and SEQ ID NO: 7). More preferably, the DNA sequence comprises 14 guanine (G14) nucleotides (SEQ ID NO: 7). In some embodiment, the DNA sequence further comprises one or more repeat sequences flanking at one or two ends of the sequence. In some embodiments, the repeat sequence is an inverted repeat, mirror repeat or direct repeat. Preferably, the DNA sequence further comprises two or more inverted repeats or direct repeats flanking two ends of the sequence.

In another aspect, the invention provides a recombinant DNA sequence, comprising a polynucleotide sequence of interest and a DNA sequence of the invention, wherein the DNA sequence is integrated into an upstream site of the polynucleotide sequence of interest.

In another aspect, the invention provides a mutator insertion sequence with integration of one or more the DNA sequence of the invention. Any mutator insertion sequence can be integrated with one or more the DNA sequence of the invention. The mutator insertion sequence refers to a recognition sequence or a recombination site that is stably integrated into the genome of a host cell. In particular, the recognition sequence or recombination site is inserted into the host genome at one or more native chromosome insertion sites present in several genes. The mutator insertion sequence may comprise regions of nucleotide sequence comprising nucleotide sequences substantially lacking homology with the genome of the host cell (e.g., randomly-generated sequences) flanking binding sites for DNA-binding domains. The DNA-binding domains that target the binding sites of the mutator insertion sequence may naturally include DNA-cleaving functional domains or may be part of fusion proteins that further comprise a functional domain, for example an endonuclease cleavage domain or cleavage half-domain (e.g., a targeting endonuclease, a recombinase, a transposase, or a homing endonuclease, including a homing endonuclease with a modified DNA-binding domain).

In one embodiment, the mutator insertion sequence comprises a ccdB gene and a DNA sequence of the invention, wherein the DNA sequence inserts into the ccdB gene at a site 30 bp from the end of the gene. In a further embodiment, the ccdB gene is further followed by another DNA sequence of the invention and another 30 bp of sequence encoding the last 10 amino acids of the ccdB protein, but using alternative codons. According to the embodiments of the invention, the DNA sequence comprises 11, 12, 13 or 14 guanine nucleotides (respectively corresponding to SEQ ID NO: 10, SEQ ID NO: 9, SEQ ID NO: 8 and SEQ ID NO: 7). More preferably, the DNA sequence comprises 14 guanine (G14) nucleotides (SEQ ID NO: 7).

In a further embodiment, the mutator insertion sequence further comprises one or more repeat sequences and optional one or more restriction enzyme sites flanking one or two ends of the open reading frame of interest of the mutator insertion sequence. In one embodiment, the repeat sequence is an inverted repeat, mirror repeat or direct repeat. In one further embodiment, at least one restriction enzyme site flanks the open reading frame of interest for a type IIS enzyme, e.g. MME1, such as restriction enzymes that generate ends outside of their recognition site, including by not limited to AarI, AceIII, AloI, BaeI, Bbr7I, BbvI, BbvII, BccI, Bce83I, BceAI, BcgI, BciVI, BfiI, BinI, BplI, BsaXI, BscAI, BseMII, BseRI, BsgI, BsmI, BsmAI, BsmFI, Bsp24I, BspCNI, BspMI, BsrI, BsrDI, BstF5I, BtgZI, BtsI, CjeI, CjePI, EcuI, Eco32I, Eco57I, Eco57MI, Esp3I, FalI, FauI, FokI, GsuI, HaelV, HgaI, Hin4I, HphI, HpyAV, Ksp632I (EarI), MME1, MboII, MlyI, MnlI, PleI, PpiI, PsrI, RleAI, SapI, VapK32I, SfaNI, SspD5I, Sth132I, StsI, TaqII, TspDTI, TspGWI, TspRI, Tth111II, as well of isoshizomers thereof. The inverted repeat allows the recognition of the mutator insertion sequence by transposase enzymes. Transposases will insert the landing pad into any DNA sequence. The transposon adaptability would allow for the insertion of the mutator insertion sequence into either a single target site, or an entire library of DNA fragments, allowing systems level scaling of the mutagenesis system of the invention. The sequence, 5-agaccggggacttatcaTccaacctgt-3′ (SEQ ID NO: 12), is one example of the inverted repeat, which provides by way of illustration only and not by way of limitation.

The direct repeat is a type of genetic sequence that consists of two or more repeats of a specific sequence are nucleotide sequences which presents in multiple copies in the genome. A direct repeat occurs when a sequence is repeated with the same pattern downstream. There is no inversion and no reverse complement associated with a direct repeat. The nucleotide sequence written in bold characters signifies the repeated sequence. The sequence, 5′-GGGGGGGGGGGGGG-3′ (SEQ ID NO: 7), is one example of the direct repeat, which provides by way of illustration only and not by way of limitation.

A DNA mirror repeat is a sequence segment delimited on the basis of its containing a center of symmetry on a single strand and identical terminal nucleotides.

Restriction enzyme sites may be introduced flanking a mutator insertion sequence to enable cloning of the mutator insertion sequence into an appropriate vector. Restriction enzyme sites may also be introduced flanking an mutator insertion sequence that produce compatible ends upon restriction enzyme digestion, to allow chaining of mutator insertion sequences together in the host genome. Restriction enzyme sites may also be introduced to allow analysis in the host of nucleic acid sequences of interest subsequently targeted to the mutator insertion sequences by recombination. Two or more restriction enzyme sites may be introduced flanking a single mutator insertion sequence. Restriction enzyme sites may also be introduced to allow analysis in the host of nucleic acid sequences of interest targeted to the mutator insertion sequence for insertion by recombination.

In an embodiment of the invention, the mutator insertion sequence comprises a ccdB gene with an insertion of a G14 repeat sequence into 30 bp from the end of the ccdB gene, then followed by a G14 repeat sequence and a sequence of final 30 bp of the ccdB gene, wherein the mutator insertion sequence further comprises one or more inverted repeats, mirror repeats or direct repeats and one or more restriction enzyme sites flanking the entire mutator insertion sequence. In a further embodiment of the invention, the mutator insertion sequence comprises a ccdB gene with an insertion of a G14 repeat sequence into 30 bp from the end of the ccdB gene, then followed by a G14 repeat sequence and a sequence of final 30 bp of the ccdB gene, wherein the mutator insertion sequence further comprises two inverted repeats, mirror repeats or direct repeats and two mme1 restriction enzyme sites flanking the entire mutator insertion sequence. In a preferred embodiment, the mutator insertion sequence is shown below.

In another aspect, the invention provides a vector comprising the DNA sequence of the invention or a mutator insertion sequence of the invention. In a further aspect, the invention provides a host cell comprising the vector of the invention.

For inserting a mutator insertion sequence into the genome of a host cell, the polynucleotide described above is typically present in a vector (“inserting vector”). These vectors are typically circular and linearized before used for recombination. In addition to the mutator insertion sequence, the vectors may also contain markers suitable for selection or screening, an origin of replication, and other elements. For example, the vector can contain both a positive selection marker and a negative selective marker. The positive screening marker is used to identify host cells into which the vector has stably integrated. The negative screening marker is used to identify cells that have randomly integrated the vector sequence.

Also provided are recombinant or engineered host cells containing a mutator insertion sequence, which are stably integrated into the genome at one or more of the native chromosomal integration sites disclosed herein. Engineered host cells can also include cells which bear such mutator insertion sequence and which then have one or more genes integrated into the mutator insertion sequence. Using the inserting vectors described above, various cells can be modified by inserting mutator insertion sequences at one or more of the specific chromosome locations.

In another further aspect, the invention provides a method for increasing a mutation rate, comprising integrating a DNA sequence or mutator insertion sequence into a gene. The method causes increases in the substitution rate downstream of the DNA sequence or mutator insertion sequence. The method provides stable, highly targeted, mutation rate increase with only the investment of a single cloning step (such as transposon-based cloning step), and the potential to introduce locally increased mutation rate to whole-systems approaches for the first time.

It is becoming increasingly clear that much mutation rate variation is due to intrinsic elements of the genome itself, and therefore should be predictable from known quantities, such as DNA sequence or chromatin composition. Accordingly, the invention is to understand these factors so that informed predictions can be made regarding the functional significance of mutations. The invention performs experiments demonstrating that simple guanine repeats increase the substitution rate up to 4 fold in the downstream kb of DNA sequence. The invention shows that the guanine repeat mutagenicity results from the interplay of both error-prone translesion synthesis (TLS) and homologous recombination repair (HR) pathways. The invention also finds that substitutions are more enriched in sequences surrounding guanine repeats and that guanine repeats are overrepresented in human genes demonstrated to be drivers of carcinogenesis.

### EXAMPLE

### Materials and Methods

Strain Construction.

All strains were constructed in a strain isogenic with W303 (MATa his3-11,15 leu2-3,112 trp1-1 ura3 ade2-1). Homopolymeric nucleotide strains were constructed by amplifying URA3, with primers containing a homopolymeric nucleotide tract at the position between -4 and -5 of URA3, the resultant PCR product transformed into ura-yeast cells using the LiAc transformation method. The URA3 gene of transformants was amplified using PCR and the sequences were confirmed by Sanger sequencing. Different mutant strains were constructed by amplifying the G418 insertion mutant for each gene of interest from the whole genome deletion collection. Strains were transformed with PCR products and deletion mutants selected for by resistance to G418. G14-repeat was constructed using an alternative URA3 sequence, which has slightly reduced function compared to the wild type URA3 gene. Change in repeat length from G14 to G15 in the G14-repeat construct reduced protein translation such that cells containing this mutation were 5- FOA resistant and detectable using the mutation rate assay.

Fluctuation Assays.

Strains to be assayed were grown overnight in 3 ml CSM-URA medium, diluted 10-4 349 and then inoculated into 100 μl cultures so that there were approximately 1000 cells per culture. At least 24 independent cultures were used per assay, and each assay repeated at least three times. Cultures were left over night at 30° C. until the cultures were assessed to have reached a suitable density, and then the entire culture, except for 5 μl, was plated onto pre-dried 5-FOA plates to detect ura3 mutants that were 5-FOA resistant. The remaining culture was pooled, diluted, and then the cell count assayed using a Scepter cell counter. Mutation rates were calculated using the maximum likelihood method [40]. In order to measure the background mutation rate at a site distal from the URA3 locus, strains were transformed with a plasmid containing an inactivated ClonNAT gene. To make the plasmid, a ClonNAT resistance gene (NATMX4) from pFA6a-NATMX4 was cloned into pRS413 using BamHI/EcoRI sites. The ClonNAT gene was engineered to include a frameshift that inactivates the gene. A frameshift causes activation of clonNAT gene and confers resistance to Nourseothricin. Cells were treated as for the URA3 mutation rate assay above, except instead of plating on 5-FOA, cells were plated on YPD plates containing Nourseothricin.

Bioinformatic and Statistical Analysis.

The genome accession numbers for Yeast and E. coli strains can be found in Table S3. Sequence and variant data for 1000 humans was downloaded from (http://www.1000genomes.org/data;ftp://ftp-trace.ncbi.nih.gov/1000genomes/ftp/release/20100804/supporting/AFR.2of4intersection_allele_f req.20100804.sites.vcf.gzftp://ftp-trace.ncbi.nih.gov/1000genomes/ftp/release/20100804/supporting/ASN.2of4intersection_allele_f req.20100804.sites.vcf.gzftp://ftp-trace.ncbi.nih.gov/1000genomes/ftp/release/20100804/supporting/EUR.2of4intersection_allele_f req.20100804.sites.vcf.gz.)

In order to identify homopolymeric guanine repeat sequences and their surrounding regions, genome sequences were aligned using BLAST with default parameters and divided into orthologous regions of at least 3 kb in length and >95% nucleotide sequence identity. Any region that could be aligned to multiple locations was not considered for analysis, ensuring that only orthologous sequences were used. A program was written in Perl script to find G13+ sequences (repeats of 13 guanines or longer), within orthologous regions; those regions not containing G13+ sequences were discarded. Nucleotide diversity was the calculated as the number of polymorphisms [41] per window of sequence. Window 1 was the first 50 bp of sequence next to the G13+ sequence, then each window after that was 100 bp. Figures were plotted using values of nucleotide diversity normalized by the average or background level of diversity as 15 calculated as the mean diversity in all windows. For calculating nucleotide 378 diversity around G379 quadruplexes, predicted G-quadruplex forming sequences were identified using “Quadparser” [42] which incorporated sequence conservation across the S. cerevisiae and S. paradoxus species listed in supplementary Table 3. The multiple alignments used to predict G quadruplexes were exploited to obtain the flanking sequences and the number of substitutions and indels counted to generate estimates of nucleotide diversity in regions surrounding G-quadruplexes in 50 bp intervals. Lists of genes experimentally verified as cancer drivers were obtained from the COSMIC census (http://cancer.sanger.ac.uk/cancergenome/projects/census/).

DNA Synthesis Stop Assay.

In order to determine whether our G11-14 sequences could form G-quadruplex structures, we conducted experiments comparing a known G-quadruplex forming sequence from Tetrahymena (GGGTTGGGTTGGGTTGGGTT) (SEQ ID NO: 13) [38] to G11, G12, G13 and G14 sequences. We designed oligonucleotides comprised of either homopolymeric runs of 11 to 14G's in a row, or the G-quadruplex sequence, integrated into the sequence context as the genomes used in this study. Following Han and co-workers [Han H, Hurley L H, Salazar M (1999) A DNA polymerase stop assay for G-quadruplex-interactive compounds. Nucleic Acids Res 27: 537-542], A radiolabelled primer (γ-³²P), shown below, was annealed with template DNA (10 nM) in buffer containing 5 mM KCl. In order to initiate the sequencing reactions MgCl (3 μM), Taq Polymerase (2.5 U per reaction) and dNTP's (final conc. 100 μM) were added and the mix incubated at either 37° C. or 55° C.

The reactions were stopped, and then run on 12% polyacrylamide gel. If the template forms a G quadruplex then DNA synthesis will not be completed, and no band can be visualized on the polyacrylamide gel.

(SEQ ID NO: 14)
Primer-[CTGCACAGAACAAAAACCTGCAGGAAACG]
Templates:
G-quadruplex control
(SEQ ID NO: 15)
GCTTTCGACATGATT(GGGTTGGGTTGGGTTGGGTT)
TATCTTCGTTTCCTGCAGGTTTTTGTTCTGTGCAG
(SEQ ID NO: 16)
G11-d[GCTTTCGACATGATTGGGGGGGGGGGTATCTT
CGTTTCCTGCAGGTTTTTGTTCTGTGCAG
(SEQ ID NO: 17)
G12-GCTTTCGACATGATTGGGGGGGGGGGGTATCTTC
GTTTCCTGCAGGTTTTTGTTCTGTGCAG
(SEQ ID NO: 18)
G13-GCTTTCGACATGATT GGGGGGGGGGGGGTATCT
TCGTTTCCTGCAGGTTTTTGTTCTGTGCAG
G14-412
(SEQ ID NO: 19)
GCTTTCGACATGATTGGGGGGGGGGGGGGTATCTTCGT
TTCCTGCAGGTTTTTGTTCTGTGCAG

### Circular Dichroism

Following [Dexheimer T S, Sun D, Hurley L H (2006) Deconvoluting the structural and drug-recognition complexity of the G-quadruplex-forming region upstream of the bcl-2 P1 promoter. J Am Chem Soc 128: 5404-5415], we incubated cuvettes containing 5 μM of oligomer DNA dissolved in Tris HCL (50 mM, pH 7.6) containing either 100 mM KCl or 100 mM NaCl for 5 minutes at 90° C., and then let them slowly cool to 25° C. Circular dichroism spectra were measured on a spectropolarimeter (J-815, JASCO, Japan) using a 1 cm path length quartz cuvette, over a range of 200-320 nm, with a response time of 1 s and a scanning speed of 100 nm.min-1. Three replicate measurements were taken, measured at 25° C.

### Example 1

### Homopolymeric Runs of Guanines 13 bp or Longer (G13+) Cause an Increase in Mutation Rate

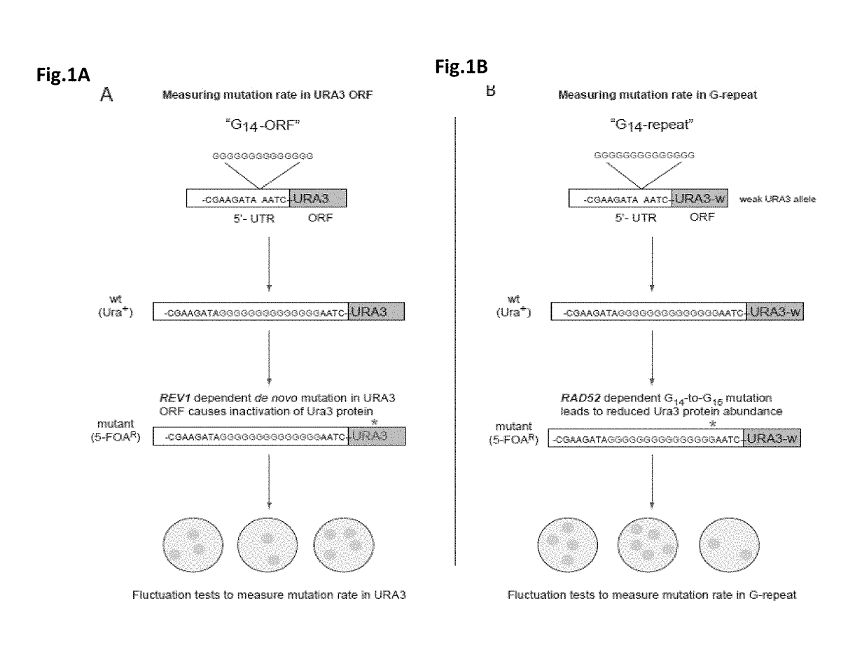

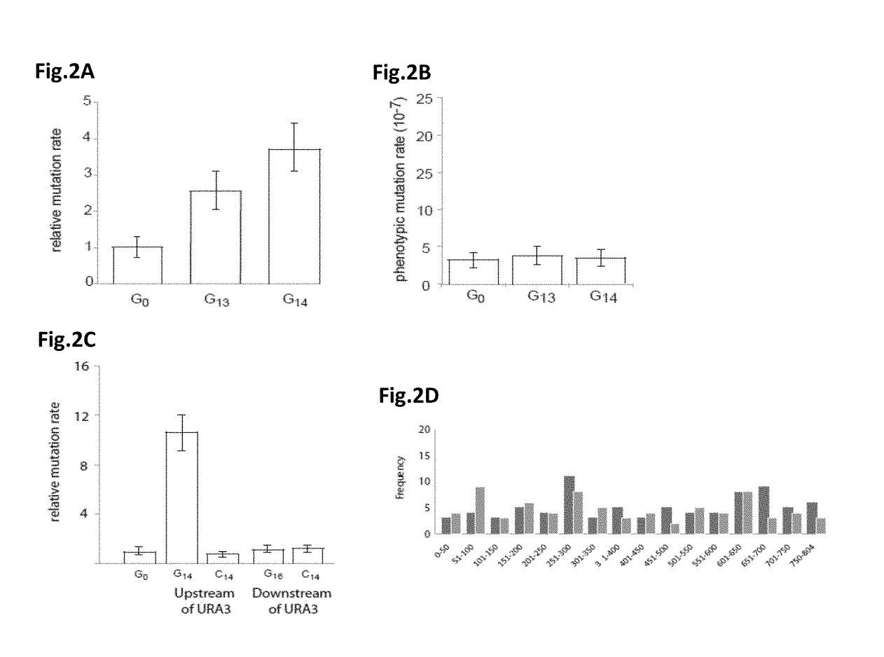

We engineered runs of 11 to 14 guanine nucleotides four bases upstream of the URA3 coding region (FIG. 1A) in Saccharomyces cerevisiae and measured mutation rates (FIG. 2A). The results show that mutation rate in the URA3 coding region, downstream of a G13 or G14 repeat sequence (from now on referred to as G13+), increases by up to 4 fold. A control experiment was performed, measuring the mutation rate at another locus (FIG. 2B), confirming that mutation rates obtained at a site that did not have a G13+ sequence upstream were indistinguishable from the wild type. This establishes that the G13+ mediated increase in mutation rate is not genome-wide but is localized to URA3. The construction of G repeats at different positions relative to the coding sequence showed that the mutagenic effect of the G14 sequence depends on whether it is present on the coding strand as changing the G repeat from the coding to template strand abolished the mutagenic effect. Moreover, moving the G14 sequence from upstream of the URA3 sequence to downstream, just after the URA3 stop codon, also removed the mutagenic effect (FIG. 2C).

The sequencing of 113 independent G14-ORF ura3 mutants established that the mutation sites were distributed relatively evenly across all 804 nucleotides of the URA3 coding region (FIG. 2D), and that changes in guanine repeat length (repeat expansion or contraction), and large deletions were not responsible for any of the elevated mutation rate detected in this assay (Table S1). Moreover, comparison of these 113 mutants to the sequences of 101 G0 ura3 mutants obtained in this study and 201 ura3 mutants obtained in another study [Tang W, Dominska M, Gawel M, Greenwell PW, Petes T D (2013) Genomic deletions and point mutations induced in Saccharomyces cerevisiae by the trinucleotide repeats (GAA.TTC) associated with Friedreich's ataxia. DNA Repair (Amst) 12: 10-17] found an equal amount of overlap in the sets of mutated sites, supporting that the insertion of the G14 does not increase the mutation rate by increasing the number of potential loss-of-function mutations. It is interesting that the mutational spectrum 126 does not seem to change between G14 and G0, suggesting that the same mutational processes are going on, but that the processes that result in mutation are induced at a higher rate by the G14 sequence.

TABLE S1
Summary of mutations from sequencing ura3 mutants
in the G₀, G₁₄ or G₁₄-repeat strains.

| | substitution | | Indel in | Indel in | a.a. | Colonies with | Total |
|---|---|---|---|---|---|---|---|
| | Transv. | Transit. | ORF | polyG | change | mutations | Colonies No. |
| G0 | 55 | 24 | 10 | N/A | 88 | 83 | 101 |
| G14 | 40 | 30 | 16 | 2 | 85 | 79 | 113 |
| G14- | 6 | 4 | 0 | 98 | 9 | 101 | 104 |
| repeat | | | | | | | |

| | | G0 | G14 | G14-repeat |
|---|---|---|---|---|
| Transversion | A −> T | 4 | 4 | 0 |
| | A −> C | 5 | 2 | 2 |
| | T −> A | 1 | 4 | 0 |
| | T −> G | 3 | 3 | 1 |
| | G −> T | 11 | 11 | 1 |
| | G −> C | 18 | 10 | 1 |
| | C −> G | 5 | 3 | 0 |
| | C −> A | 8 | 3 | 1 |
| Transition | A −> G | 1 | 2 | 2 |
| | T −> C | 7 | 6 | 1 |
| | G −> A | 10 | 11 | 1 |
| | C −> T | 6 | 11 | 0 |

### Example 2

### G13+ Repeats are Under-represented in Genomes and Over-represented in Somatically Mutated Cancer Cells

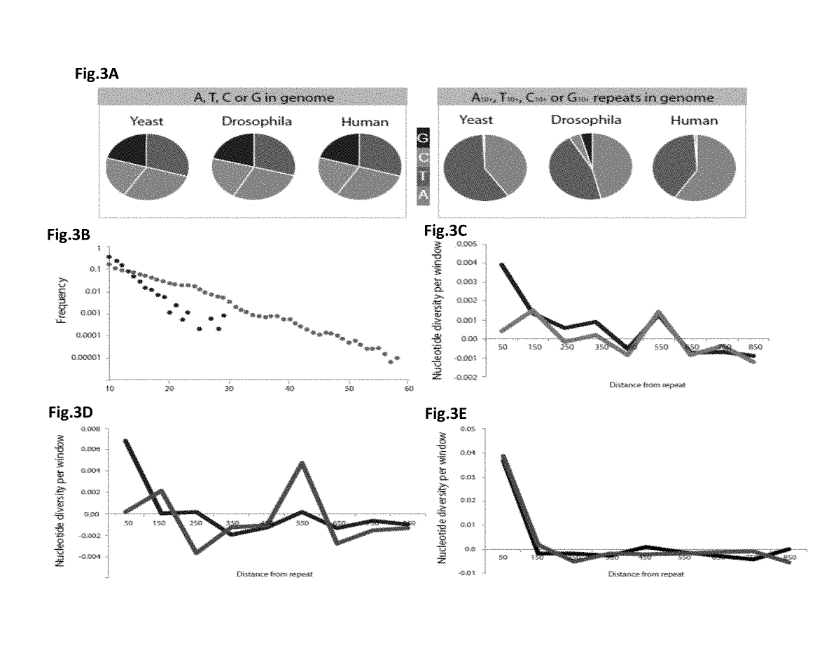

Most de novo mutations are deleterious [Keightley P D, Lynch M (2003) Toward a realistic model of mutations affecting fitness. Evolution 57: 683-685]. As such, evolutionary theory predicts that sequences that cause an elevation in mutation rates should suffer attrition by purifying selection due to an increased likelihood of linkage with deleterious mutations. An expected consequence of such purifying selection is that homopolymeric guanine repeat sequences should be less common in genomes than expected. In order to investigate this we examined multiple individual genomes within E. coli, yeast and Human. While, the ratios of the total amount of A, T, C and G nucleotides are distributed as expected (FIG. 3A), we found that C10+ and G10+ homopolymeric repeats of 10 nucleotides or more, are drastically depleted when compared to T10+ and A10+ sequences (FIG. 3A). E. coli and Yeast genomes typically had only 1 G10+ repeat. While Humans had many more of all kinds of repeats, human and yeast genomes were both 50× more likely to have A13 than G13 repeats, and the mean length of repeats greater than 10 nucleotides was longer for G's than A's (Humans, G=11.6 and A=14.3; yeast, G=11.3 and A=13.7 bases). Using human genome data, which had enough G and A repeats to allow for a robust comparison, we normalized the G and A distributions, finding a clear depletion of G repeats relative to A, for repeats 10 nucleotides and longer (FIG. 3B). We next looked at the substitution and indel rate in windows of sequence surrounding A and G repeats (Methods) using human 1000 genome polymorphism data. We found that while the rate of indel mutations were indistinguishable between A and T repeats, the nucleotide substitution rates were much higher in the window of sequence closest to G repeats than A repeats (Wilcoxen signed rank, coding p=0.0058, non-coding=0.0001). While the nucleotide diversity surrounding G repeats is much higher than the background, the nucleotide diversity close to A repeats showed a sporadic distribution of substitutions compared to the background rate of divergence (FIG. 3C-E).

With the knowledge the homopolymeric G repeat sequences cause a higher mutation rate, and are depleted in genomes, we next sought to investigate whether there was a biased distribution of G13+ repeats across different classes of genes. For this analysis we focused on the Cancer Gene Census [Forbes S A, Bindal N, Bamford S, Cole C, Kok C Y, et al. (2011) COSMIC: mining complete cancer genomes in the Catalogue of Somatic Mutations in Cancer. Nucleic Acids Res 39: D945-950], which provides a list of genes that have been experimentally confirmed as causal “driver” genes of carcinogenesis. We compared the list of genes that contain G13+ to two subsets of these data, genes mutated during somatic clonal evolution of the cancer, and genes in which mutation causes a hereditary predisposition to cancer. We found that mutations that were acquired during the somatic progression of the cancer were significantly enriched with genes that contain G13+ sequences (hypergeometric distribution, n=483, p=1.3×10⁻⁵). In contrast, G13+ (or longer), sequences were completely absent from the list of germ-line cancer predisposition genes (n=81).

### Example 3

### Replication Timing Correlates with G13+ Mutagenicity

Replication timing is known to correlate with mutation rate variation in organisms ranging from bacteria to humans and is the strongest known correlate with mutation rate variation in cancer. Mutation rate differences are only detectable between the extremes of the replication-timing continuum, and vary on 10-100 kb scales. Repeat sequences have greater fold impact on mutation rate, 182 but across smaller scales (within 1 kb). It is of interest to investigate how the short distance effects of repeat sequences interact with the genome scale effects of replication timing, as combining these two known influences of mutation rate would further improve models of the genome-wide mutation rate landscape.

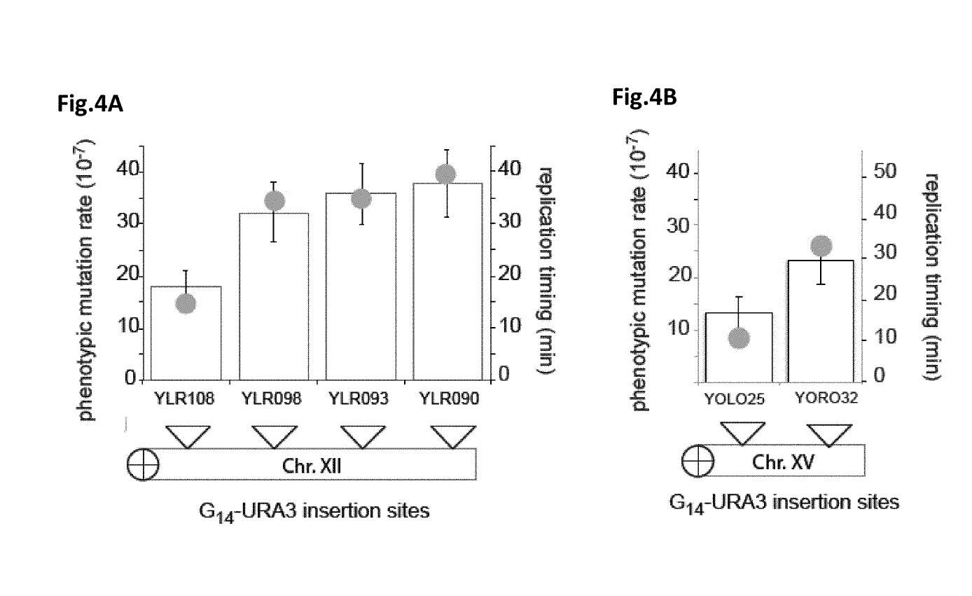

To test whether genome position would influence the G14 mutagenicity, G0-URA3 and G14-URA3 genes were engineered into different positions on chromosomes XII and XV (FIG. 4A and FIG. 4B). Increases in mutation rates similar to those observed to chromosome V (the original locus of URA3) were measured, confirming that G14 mutagenicity occurs regardless of genome position. Interestingly, differences in G14-URA3 mutation rate were observed between different positions and found to be directly proportional to DNA replication timing (R2 191=0.97, p=0.00676) (replication timing from [Nieduszynski Calif., Knox Y, Donaldson A D (2006) Genome-wide identification of replication origins in yeast by comparative genomics. Genes Dev 20: 1874-1879]). The finding that G14 mutagenicity interacts in a highly predictable manner with DNA replication timing, suggests that the mutations mainly occur during chromosome replication.

### Example 4

### Mutagenesis Downstream of G13+Repeats is Rev1 dependent

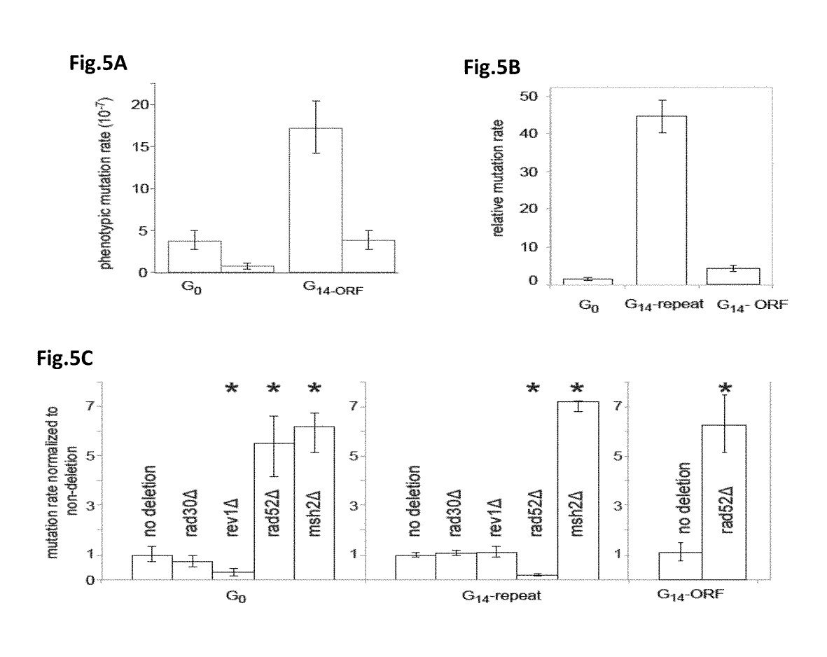

Repeat sequences are known to suffer an increased risk of replication fork stalling. Upon fork stalling, replication reinitiates downstream, leaving a single stranded gap that is filled in using either homologous recombination (HR) or translesion synthesis (TLS), with a bias towards TLS for gaps requiring repair later in S phase. Previously we had proposed that repeat sequence-mediated increases in downstream mutation rate were caused by frequent recruitment of error-prone translesion DNA polymerases by sequences prone to stall the high-fidelity, housekeeping DNA polymerase. To test this, we deleted REV1, an essential component for error prone repair, forming a complex with polymerase polζ required for all TLS in yeast. We found that ablation of REV1 significantly reduced the mutation rate of both G14-ORF (t test, p<0.005) and G0 (t-test, <0.005) (FIG. 5A). The reason that deletion of REV1 decreases mutation 211 rate, is that the replication fork interruptions that are typically accommodated by Rev1-mediated TLS DNA synthesis are lethal in the rev1 mutant. This result suggests that G13+ sequences cause an increased likelihood of mutation in the surrounding DNA sequence by increasing the rate of error-prone translesion DNA synthesis. However, the G14 rev1 mutation rate was significantly higher than the G0 mutation rate (t=31, <0.005) (FIG. 5A), suggesting that a small component of G14-mediated increase in mutation rate is Revl independent.

### Example 5

### Expansion of Homopolymeric Repeats Also Occurs at the G13+ Repeat and is Rad52 Dependent

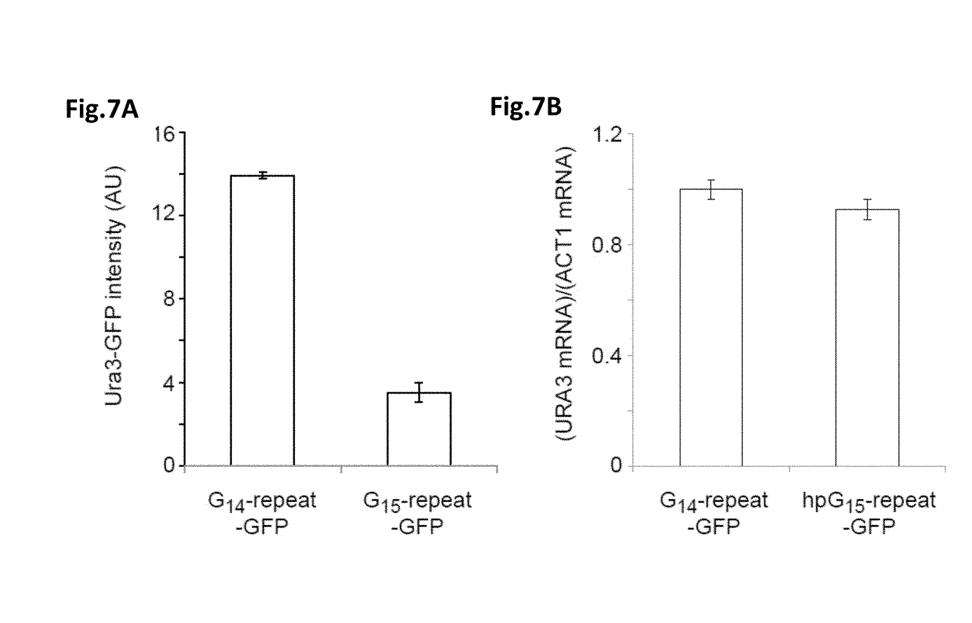

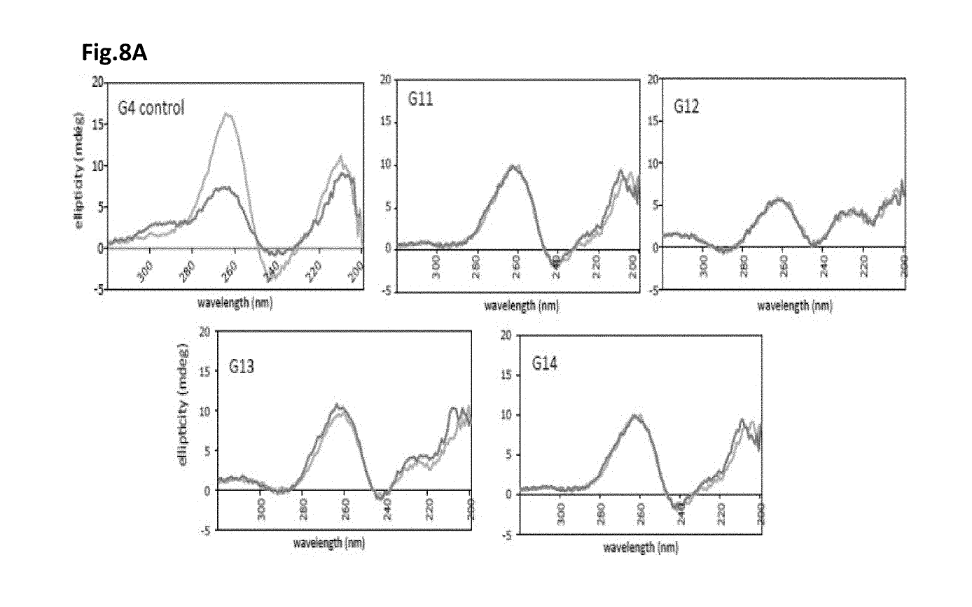

It has long been established that homopolymeric repeat sequences are unstable, increasing and decreasing in repeat length at a high rate. In the experiment described above, only mutations that occur in the open reading frame of URA3 can be recovered by the screen, even though repeat length change mutations almost certainly occur in the G14 sequences of some of the individuals within the large yeast populations used to measure mutation rate. This is because mutations changing the length of the G14 repeat, which is in the 5′ UTR region, do not cause the loss of URA3 function that the assay selects upon. In order to facilitate the capture of mutations changing the number of G's in the G14 repeat, a new strain was constructed containing the G14 sequence, this time engineered upstream of an alternative URA3 sequence (URA3-w), whose function is mildly compromised (G14-repeat, FIG. 1B). We had previously observed that a G15 repeat in 5′ UTR region of URA3-w could cause a reduction in protein translation (FIG. 7A and FIG. 7B). Although G14-URA3-w exhibits the Ura+ 237 phenotype of the wild type allele, a mutation from G14 to G15 (or longer) results in the assayable loss of URA3 function, probably 238 due to a combined effect of impaired function and reduced translation (FIG. 1B). This construct (G14-repeat) was used to directly measure the mutation rate of repeat length increase from G14 to G15 or longer (contraction of the G repeat would not generate the phenotype). Repeat length-dependent increase in mutation rate was found to be much higher than the downstream mutation rate, with a 45 fold difference between G14-repeat and G0 cells (FIG. 5B). Sequencing 105 independent ura3 mutant clones of G14-repeat confirmed that the majority of them carried an increased polyguanine repeat (G15) but no mutations in the coding region and no large deletions (Table 51).

While deletion of REV1 reduced the mutation rate within the URA3 ORF as detected by the G14-ORF construct, deletion of REV1 had no effect on the mutation rate in the G-repeat as measured using the G14-repeat construct (FIG. 5C, 2^{nd} box). To confirm that another translesion DNA synthesis pathway was not involved, a gene essential for another translesion pathway, RAD30, was deleted, also having no effect on mutation rate. We next turned to the alternative mechanism for rescue of the stalled replication fork, homologous recombination. Rad52 is essential for the annealing of DNA strands during homologous recombination, and its ablation causes an increase in mutation rate of approximately 5 fold in G0 cells (FIG. 5C). This reason for this increase is that rad52 mutants depend upon error-prone DNA olymerases to synthesize over the single stranded gaps resulting from replication fork stalling. Conversely, we found that deletion of RAD52 in the G14-repeat strain reduced URA3 mutation rates dramatically (FIG. 5C), consistent with previous work examining recombination and frameshifts underlying “adaptive mutation” in E. coli. We checked whether rad52 deletion was able to reduce the mutation rate in G14-ORF cells. However, similarly to G0 cells, the mutation rate was increased approximately 5 fold (FIG. 5C, 3rd box), showing that Rad52-mediated homologous recombination effects only the change of G14 length, not the downstream mutagenic effect of G14. MSH2 deletion mutants were also measured for the G0 and G14-repeat strains to check for any interaction between the mismatch repair pathway and G14 mutagenesis.

However, MSH2 deletion increased mutation rate by the same degree in both strains, as expected from previous studies [Drotschmann K, Clark A B, Tran H T, Resnick M A, Gordenin D A, et al. (1999) Mutator phenotypes of yeast strains heterozygous for mutations in the MSH2 gene. Proceedings of the National Academy of Sciences of the United States of America 96: 2970-2975].

### Example 6

### G13+ Mutagenesis is Not Caused by Formation of G-quadruplex Structures

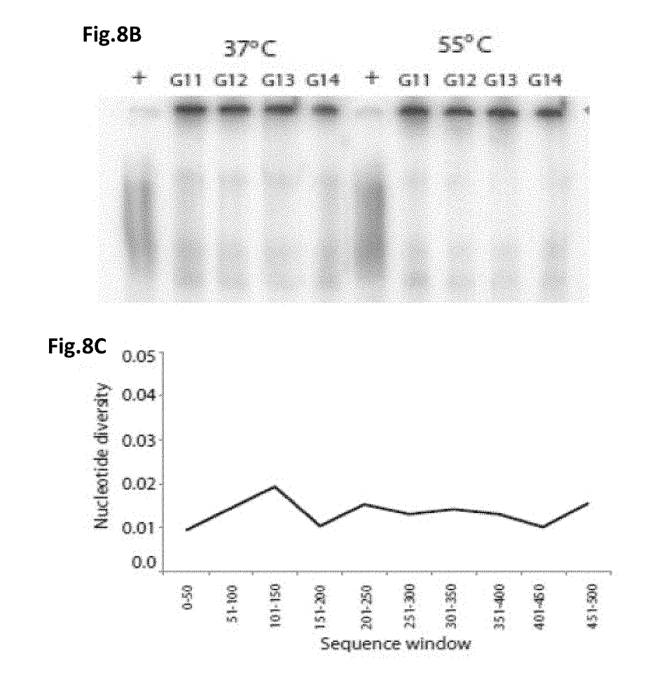

Double strand breaks have been shown to be mutagenic towards surrounding 266 DNA sequence. Here, the dependence of downstream mutagenesis on Revl, and its independence from Rad52 are strong evidence that G14-mediated mutagenesis is not due to double strand break repair, but rather that G14 may impede the replication fork. Moreover, when the URA3 gene was PCR amplified from 113 independent ura3 mutant clones of G14, a PCR product of the predicted size was obtained in all clones, as well as complete DNA sequences, indicating that large deletions, a tell tale sign of double strand break repair, had not occurred in the mutant clones. However, it is plausible that G14 sequences could form into G-quadruplex structures, which can cause the replication fork to pause and may promote genetic instability. In order to test whether our polyguanine sequences could form G-quadruplex structures, we conducted experiments comparing a known G-quadruplex forming sequence from tetrahymena to G11, G12, G13 and G14 sequences. We designed 5 oligomers (given in Materials and Methods) that included either the G-quadruplex control sequence, or 11 to 14 Guanines's in a row, each integrated into the same sequence context as the URA3 constructs used for fluctuation tests in this study. We first performed Circular Dichroism analysis of the oligos in ionic solutions that support the folding of G quadruplex, confirming that the control did indeed form a G-quadruplex in the test conditions (FIG. 8A).

We then performed DNA polymerase stop assays to find whether DNA polymerase could synthesize the complementary DNA across the single stranded template, based on the principle that a stable secondary structure should inhibit DNA synthesis. The results show that while a known G-quadruplex structure blocked the polymerase, G11-G14 sequences did not have the same effect (FIG. 8B).

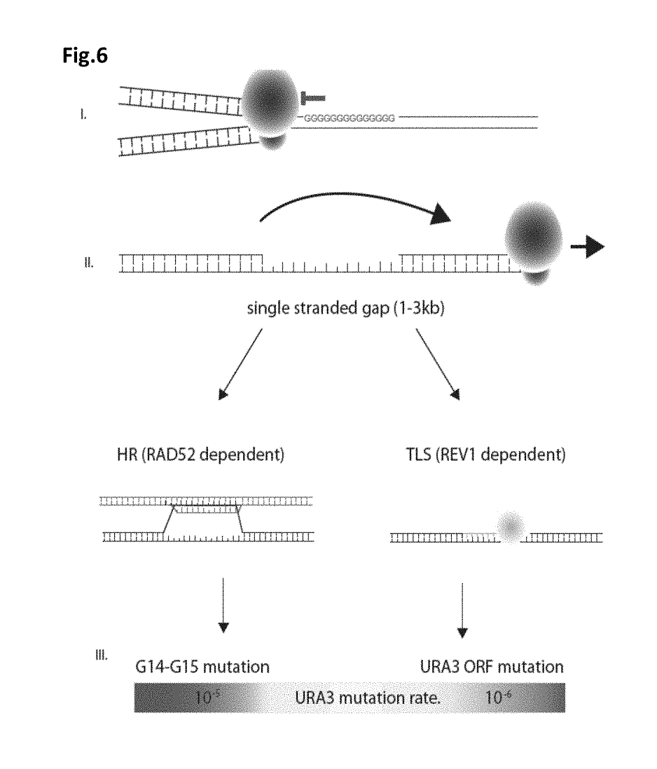

Our experimental confirmation that G13+ induced mutation correlates with replication timing supports that the repair mechanism of choice is S phase dependent. Further, the two constructs, G14 294 and G14-repeat, allow for the parsing of the two repair mechanisms at G13+ sequences; Revl-mediated bypass, most likely resulting in elevated downstream mutation rates, and Rad52-mediated homologous recombination, most likely resulting in repeat length change. Although Rad52-mediated homologous recombination is generally not considered to be mutagenic, error rates during HR have been shown to be higher than during normal S phase DNA replication. Here the homologous repair error rate downstream of the G14 sequence is extremely low, however the mutation rate in the repeat sequence is magnitudes higher than Revl-mediated DNA synthesis. These results provide a glimpse of multiple DNA replication and repair processes acting upon a difficult-to-replicate element of DNA sequence (FIG. 6).

What is claimed is:

1. A DNA sequence, comprising a short repeat nucleotide sequence of less than 20 guanine or adenine.

2. The DNA sequence of claim 1, which comprises 20 (SEQ ID NO:1), 19 (SEQ ID NO:2), 18 (SEQ ID NO:3), 17 (SEQ ID NO:4), 16 (SEQ ID NO:5), 15 (SEQ ID NO:6), 14 (SEQ ID NO:7), 13 (SEQ ID NO:8), 12 (SEQ ID NO:9), 11 (SEQ ID NO:10) or 10 (SEQ ID NO:11) guanine nucleotides.

3. The DNA sequence of claim 1, which comprises 11 (SEQ ID NO:10), 12 (SEQ ID NO:9), 13 (SEQ ID NO:8) or 14 (SEQ ID NO:7) guanine nucleotides.

4. The DNA sequence of claim 1, which comprises 14 guanine nucleotides (SEQ ID NO: 7).

5. The DNA sequence of claim 1, which further comprises one or more repeat sequences flanking one or two ends of the sequence.

6. The DNA sequence of claim 5, wherein the repeat sequence is an inverted repeat, mirror repeat or direct repeat.

7. A recombinant DNA sequence, comprising a polynucleotide sequence of interest and a DNA sequence of claim 1, wherein the DNA sequence is integrated into an upstream site of the polynucleotide sequence of interest.

8. A mutator insertion sequence integrated with one or more the DNA sequence of claims 1.

9. The mutator insertion sequence of claim 8, which comprises a ccdB gene and the DNA sequence, wherein the DNA sequence inserts into the ccdB gene at a site 30 bp from the end of the gene.

10. The mutator insertion sequence of claim 9, wherein the ccdB gene is further followed by a DNA sequence comprising a short repeat nucleotide sequence of less than 20 guanine or adenine and another 30 bp of sequence encoding the last 10 amino acids of the ccdB protein, but using alternative codons.

11. The mutator insertion sequence of claim 8, which further comprises one or more repeat sequences and optional one or more restriction enzyme sites flanking one or two ends of the open reading frame of interest of the mutator insertion sequence.

12. The mutator insertion sequence of claim 11, wherein the repeat sequence is an inverted repeat, mirror repeat or direct repeat.

13. The mutator insertion sequence of claim 11, wherein the restriction enzyme site is mme 1 restriction enzyme site.

14. The mutator insertion sequence of claim 11, comprising a ccdB gene with an insertion of a G14 repeat sequence (SEQ ID NO: 7) into 30 bp from the end of the ccdB gene, then followed by a G14 repeat sequence (SEQ ID NO: 7) and a sequence of final 30 bp of the ccdB gene, wherein the mutator insertion sequence further comprises one or more additional repeat sequences and one or more restriction enzyme sites flanking the entire mutator insertion sequence.

15. The mutator insertion sequence of claim 14, wherein the additional repeat sequence is an inverted repeat, mirror repeat or direct repeat.

16. The mutator insertion sequence of claim 11, comprising a ccdB gene with an insertion of a G14 repeat sequence into 30 bp from the end of the ccdB gene, then followed by a G14 repeat sequence and a sequence of final 30 bp of the ccdB gene, wherein the mutator insertion sequence further comprises two inverted repeats or direct repeats and two restriction enzyme sites flanking the open reading frame of interest of the mutator insertion sequence.

17. The mutator insertion sequence of claim 14, wherein the restriction enzyme site is AarI, AceIII, AloI, BaeI, Bbr7I, BbvI, BbvII, BccI, Bce83I, BceAI, BcgI, BciVI, BfiI, BinI, BplI, BsaXI, BscAI, BseMII, BseRI, BsgI, BsmI, BsmAI, BsmFI, Bsp24I, BspCNI, BspMI, BsrI, BsrDI, BstF5I, BtgZI, BtsI, CjeI, CjePI, EcuI, Eco32I, Eco57I, Eco57MI, Esp3I, FalI, FauI, FokI, GsuI, HaelV, HgaI, Hin4I, HphI, HpyAV, Ksp632I (EarI), MME1, MboII, MlyI, MnlI, PleI, PpiI, PsrI, RleAI, SapI, VapK32I, SfaNI, SspD5I, Sth132I, StsI, TaqII, TspDTI, TspGWI, TspRI, Tth111II, or an isoshizomer thereof.

18. A vector comprising a mutator insertion sequence of claim 8.

19. A host cell comprising the vector of claim 18.

20. A method for increasing a mutation rate, comprising integrating a DNA sequence of claim 1.

21. A method for increasing a mutation rate, comprising integrating a mutator insertion sequence of claim 8.
